(19) United States
(12) Patent Application Publication
(10) Pub. No.: US 2024/0142424 A1
(43) Pub. Date: May 2, 2024

(54) HYDROGEN DETECTION SYSTEM

(71) Applicant: ROHM Co., LTD., Kyoto (JP)

(72) Inventor: Shunsuke Akasaka, Kyoto (JP)

(21) Appl. No.: 18/492,079

(22) Filed: Oct. 23, 2023

(30) Foreign Application Priority Data
Nov. 1, 2022 (JP) 2022-175702

(51) Int. Cl. G01N 33/00

(52) U.S. Cl. CPC G01N 33/005; G01N 33/0073

(57) ABSTRACT

Provided is a hydrogen detection system including a thermal conductivity type hydrogen sensor configured to detect a hydrogen concentration, a humidity sensor configured to detect humidity, and a controller. The controller corrects a first detection value detected by the thermal conductivity type hydrogen sensor, on the basis of the humidity detected by the humidity sensor, and outputs the hydrogen concentration as a second detection value.

### CROSS REFERENCE TO RELATED APPLICATIONS

This application claims priority benefit of Japanese Patent Application No. JP 2022-175702 filed in the Japan Patent Office on Nov. 1, 2022. Each of the above-referenced applications is hereby incorporated herein by reference in its entirety.

### BACKGROUND

The present disclosure relates to a hydrogen detection system.

In the past, a hydrogen sensor for detecting a hydrogen concentration has been developed. Various types of hydrogen sensor such as a semiconductor type, a catalytic combustion type, and a thermal conductivity type are known as the hydrogen sensor. Japanese Patent Laid-Open No. 2017-198541 discloses a technology for preventing a dew condensation in a semiconductor type hydrogen sensor.

### BRIEF DESCRIPTION OF THE DRAWINGS

FIG. 1 is a block diagram illustrating a configuration of a hydrogen detection system;

FIG. 2 is a circuit diagram of a thermal conductivity type hydrogen sensor;

FIG. 3 is a diagram of assistance in explaining how a relation between a hydrogen concentration sensed by the thermal conductivity type hydrogen sensor and an actual hydrogen concentration changes according to differences in dew point in a case where the temperature in the atmosphere is 20° C.;

FIG. 4 is a diagram of assistance in explaining how the relation between the hydrogen concentration sensed by the thermal conductivity type hydrogen sensor and the actual hydrogen concentration changes according to differences in dew point in a case where the temperature in the atmosphere is 40° C.;

FIG. 5 is a diagram of assistance in explaining how the relation between the hydrogen concentration sensed by the thermal conductivity type hydrogen sensor and the actual hydrogen concentration changes according to differences in dew point in a case where the temperature in the atmosphere is 60° C.;

FIG. 6 is a flowchart illustrating processing of correcting the hydrogen concentration;

FIG. 7 is a plan view of a humidity sensor in which a porous protective layer is not illustrated;

FIG. 8 is a plan view of the humidity sensor in which an insulating layer and a porous metallic layer are not illustrated;

FIG. 9 is a sectional view taken along a line IX-IX of FIG. 7; and

FIG. 10 is a sectional view of a humidity sensor according to a modification.

### DETAILED DESCRIPTION

An embodiment of the present disclosure will hereinafter be described in detail with reference to the drawings. Note that identical or corresponding parts are identified by the same reference signs in the figures, and description thereof will not be repeated.

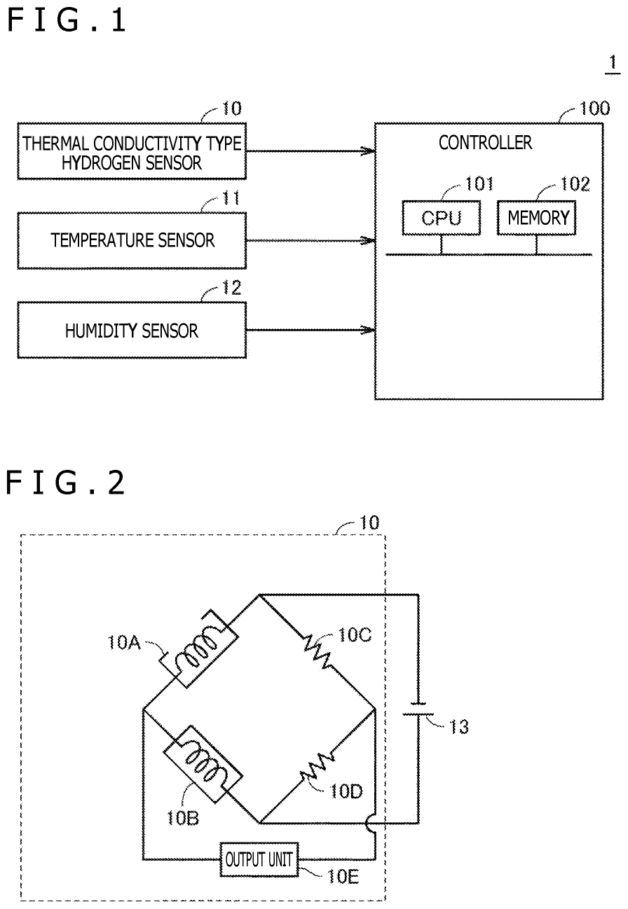

FIG. 1 is a block diagram illustrating a configuration of a hydrogen detection system 1. The hydrogen detection system 1 according to the embodiment of the present disclosure includes a thermal conductivity type hydrogen sensor 10, a temperature sensor 11, a humidity sensor 12, and a controller 100.

The thermal conductivity type hydrogen sensor 10 is a sensor for detecting a hydrogen concentration. The temperature sensor 11 is a sensor for detecting a temperature. The humidity sensor 12 is a sensor for detecting humidity. An yttria-stabilized zirconia (YSZ) humidity sensor that detects absolute humidity, for example, is used as the humidity sensor 12. Information regarding detection values detected by the respective sensors is transmitted to the controller 100 at a predetermined timing.

The controller 100 includes a central processing unit (CPU) 101, a memory 102 (a read only memory (ROM) and a random access memory (RAM)), and an input-output buffer (not illustrated), for example. The CPU 101 expands programs stored in the ROM into the RAM or other memories and executes the programs. The programs stored in the ROM are programs in which a processing procedure for the controller 100 is described. The controller 100 processes the information received from each device in the hydrogen detection system 1 according to these programs. This control is not limited to being processed by software, and can also be processed by dedicated hardware (electronic circuit).

FIG. 2 is a circuit diagram of the thermal conductivity type hydrogen sensor 10. The thermal conductivity of hydrogen is approximately seven times the thermal conductivity of the air. The thermal conductivity type hydrogen sensor 10 detects hydrogen by using a difference in thermal conductivity between hydrogen and the air. The thermal conductivity type hydrogen sensor 10 constitutes a bridge circuit including a detecting element 10A, a compensating element 10B, a resistance 10C, a resistance 10D, and an output unit 10E. The detecting element 10A has a structure that is exposed to a measurement environment and is in contact with a hydrogen gas. The compensating element 10B has a structure that is sealed with the air sealed therein and is not in contact with the hydrogen gas.

The detecting element 10A and the compensating element 10B are heated to approximately 300° C. by a predetermined voltage applied thereto from a power supply 13. In the thermal conductivity type hydrogen sensor 10, in a case where the hydrogen gas is present, a state of heat dissipation of the detecting element 10A changes due to the thermal conductivity unique to the hydrogen gas, and consequently, the temperature of the detecting element 10A changes. With this temperature change, the resistance value of a platinum wire coil constituting the detecting element 10A changes. The change in the resistance value is substantially proportional to the concentration of the hydrogen gas. The thermal conductivity type hydrogen sensor 10 extracts an amount of change in the resistance value as a voltage by the bridge circuit, and outputs the voltage from the output unit 10E. The gas concentration is thus detected.

The thermal conductivity type hydrogen sensor 10 is a physical sensor that does not involve chemical reaction. Hence, the thermal conductivity type hydrogen sensor 10 has a good tolerance to volatile siloxane generated from silicon included in a packing, a seal material, and other materials used for a fuel cell, as compared with chemical sensors of the semiconductor type, the catalytic combustion type, and other types. However, the thermal conductivity type hydrogen sensor 10 may not be able to adequately detect a low concentration of hydrogen due to an effect of the humidity. For example, in a case where the humidity is high, the hydrogen concentration sensed by the thermal conductivity type hydrogen sensor 10 is lower than an actual hydrogen concentration. This is because, in a state in which the hydrogen concentration is low and there is a lot of water vapor in the atmosphere, the thermal conductivity type hydrogen sensor 10 extracts a detected amount of water vapor as a detected amount of hydrogen.

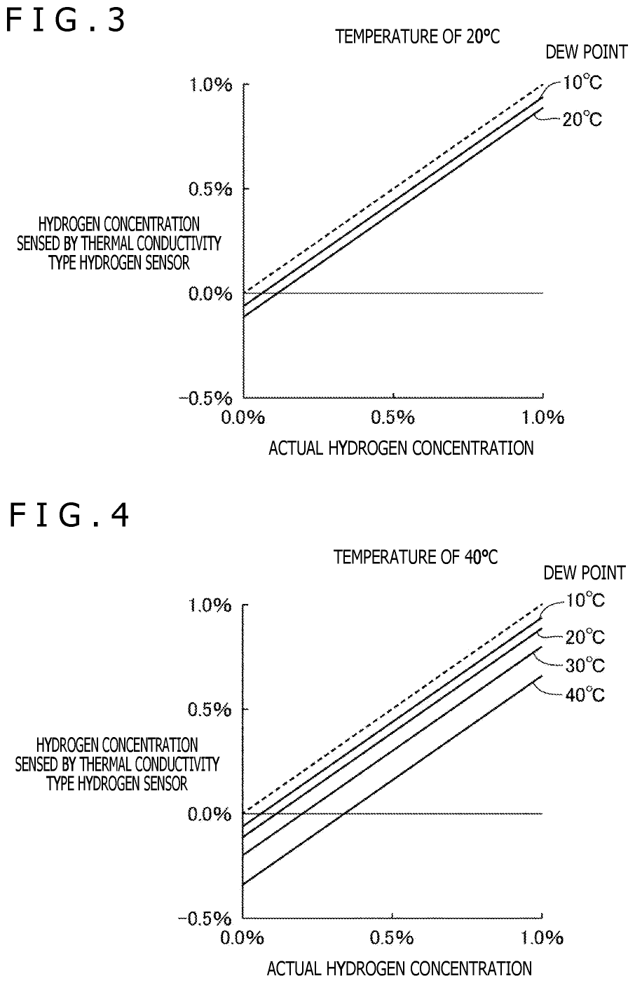

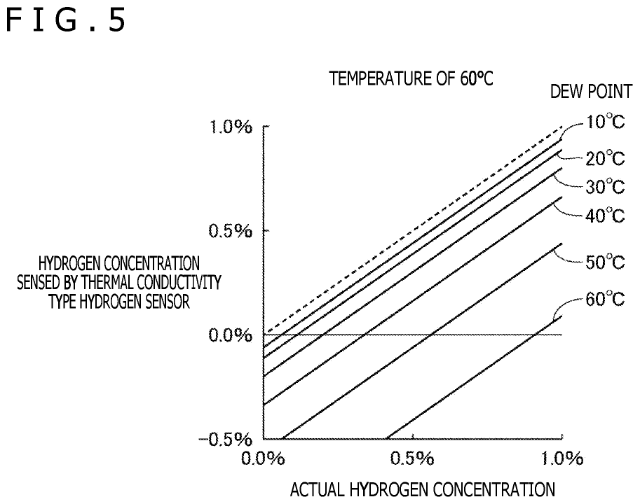

In the hydrogen detection system 1 according to the present embodiment, the hydrogen concentration sensed by the thermal conductivity type hydrogen sensor 10 is corrected to an actual hydrogen concentration by taking the effect of the humidity into consideration. FIG. 3 is a diagram of assistance in explaining how a relation between the hydrogen concentration sensed by the thermal conductivity type hydrogen sensor 10 and the actual hydrogen concentration changes according to differences in dew point in a case where the temperature in the atmosphere is 20° C. FIG. 4 is a diagram of assistance in explaining how the relation between the hydrogen concentration sensed by the thermal conductivity type hydrogen sensor 10 and the actual hydrogen concentration changes according to differences in dew point in a case where the temperature in the atmosphere is 40° C. FIG. 5 is a diagram of assistance in explaining how the relation between the hydrogen concentration sensed by the thermal conductivity type hydrogen sensor 10 and the actual hydrogen concentration changes according to differences in dew point in a case where the temperature in the atmosphere is 60° C.

The pieces of data illustrated in FIGS. 3 to 5 are stored in advance as conversion tables in the memory 102, which is a storage device of the controller 100. Although the conversion tables where the temperature in the atmosphere is 20° C., 40° C., and 60° C. will be described below, conversion tables corresponding to other temperatures are also stored in the memory 102.

In a case where the temperature in the atmosphere stays the same, there is such a relation that the higher the dew point is (the larger the amount of water vapor included in the air is), the higher the humidity is. The thermal conductivity type hydrogen sensor 10 tends to be affected by the humidity when detecting a low concentration of hydrogen. Therefore, as illustrated in FIGS. 3 to 5, the higher the dew point becomes at each temperature, the more the relation between the hydrogen concentration sensed by the thermal conductivity type hydrogen sensor 10 and the actual hydrogen concentration deviates from an ideal straight line indicated by a broken line. In particular, the hydrogen concentration sensed by the thermal conductivity type hydrogen sensor 10 considerably deviates from the actual hydrogen concentration as the dew point changes to 40° C., 50° C., and 60° C.

In the hydrogen detection system 1, the controller 100 calculates a dew point temperature from the temperature detected by the temperature sensor 11 and the absolute humidity detected by the humidity sensor 12, by computation using a conversion coefficient. Incidentally, the conversion coefficient used to convert the absolute humidity to the dew point temperature is calculated in advance from a correlation between a current value of the humidity sensor 12 and humidity. In the hydrogen detection system 1, the controller 100 selects data, which is data as illustrated in FIGS. 3 to 5, corresponding to the temperature detected by the temperature sensor 11, and determines the actual hydrogen concentration from the hydrogen concentration sensed by the thermal conductivity type hydrogen sensor 10 and the dew point temperature.

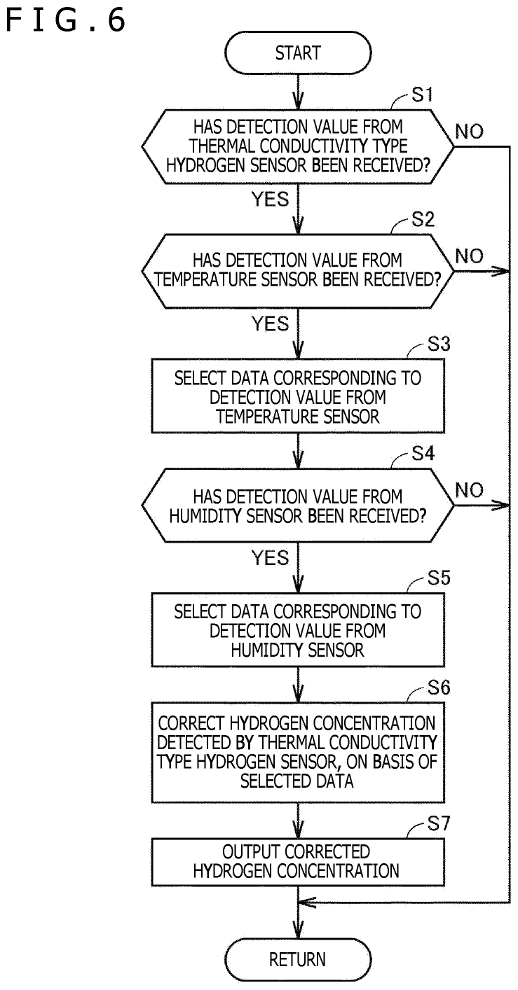

Now, processing of correcting the hydrogen concentration which is performed by the controller 100 will be described. FIG. 6 is a flowchart illustrating the processing of correcting the hydrogen concentration. The processing of FIG. 6 is repeatedly called as a subroutine from a main routine in the control performed by the controller 100, and is then executed. The controller 100 first determines in step S (hereinafter denoted simply by “S”) 1 whether or not a detection value sensed by the thermal conductivity type hydrogen sensor 10 has been received.

In a case where the controller 100 determines that the detection value sensed by the thermal conductivity type hydrogen sensor 10 has not been received (NO in S1), the controller 100 returns the processing from the subroutine to the main routine. In a case where the controller 100 determines that the detection value sensed by the thermal conductivity type hydrogen sensor 10 has been received (YES in S1), the controller 100 determines whether or not a detection value from the temperature sensor 11 has been received (S2).

In a case where the controller 100 determines that the detection value from the temperature sensor 11 has not been received (NO in S2), the controller 100 returns the processing from the subroutine to the main routine. In a case where the controller 100 determines that the detection value from the temperature sensor 11 has been received (YES in S2), the controller 100 selects data corresponding to the detection value from the temperature sensor 11, from among the pieces of data illustrated in FIGS. 3 to 5 (S3). Next, the controller 100 determines whether or not a detection value from the humidity sensor 12 has been received (S4).

In a case where the controller 100 determines that the detection value from the humidity sensor 12 has not been received (NO in S4), the controller 100 returns the processing from the subroutine to the main routine. In a case where the controller 100 determines that the detection value from the humidity sensor 12 has been received (YES in S4), the controller 100 calculates the dew point on the basis of the detection value from the humidity sensor 12 by computation, and selects data corresponding to the calculated dew point from the data selected in S3 (S5).

Next, the controller 100 corrects the hydrogen concentration detected by the thermal conductivity type hydrogen sensor 10, to the actual hydrogen concentration, on the basis of the selected data (S6). Next, the controller 100 outputs the corrected hydrogen concentration as the actual hydrogen concentration (S7). The controller 100 then returns the processing from the subroutine to the main routine.

The hydrogen detection system 1 performs the processing of correcting the hydrogen concentration detected by the thermal conductivity type hydrogen sensor 10, to the actual hydrogen concentration, as described above. By this processing, the thermal conductivity type hydrogen sensor 10 can detect low hydrogen concentrations in a range of at least 0.1% to 4%. Note that the thermal conductivity type hydrogen sensor 10 can also detect hydrogen concentrations higher than 4% but equal to or lower than 100%. Thus, the hydrogen detection system 1 according to the embodiment of the present disclosure can suitably detect a wide range of hydrogen concentrations by taking into consideration a detection error made by the thermal conductivity type hydrogen sensor 10 due to the humidity.

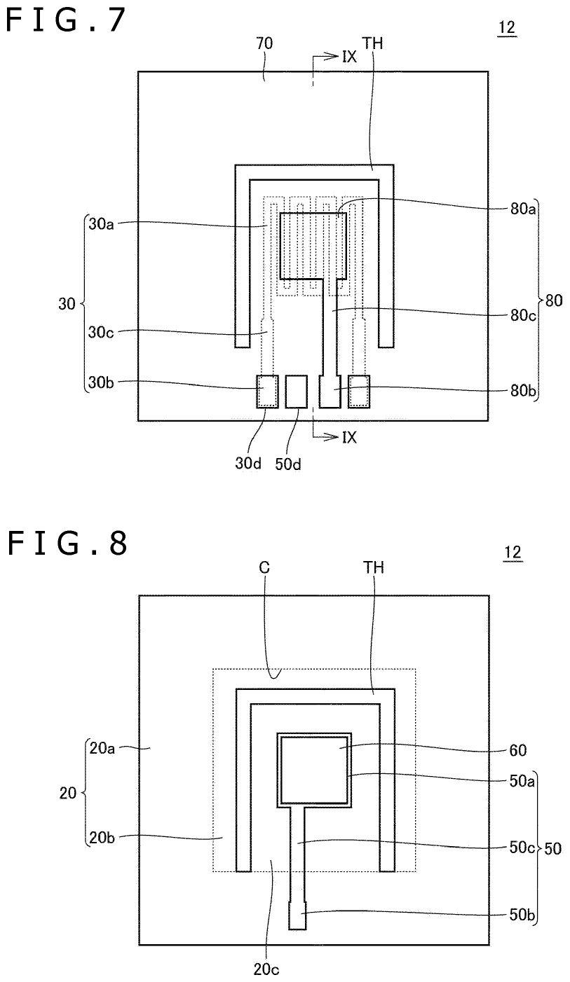

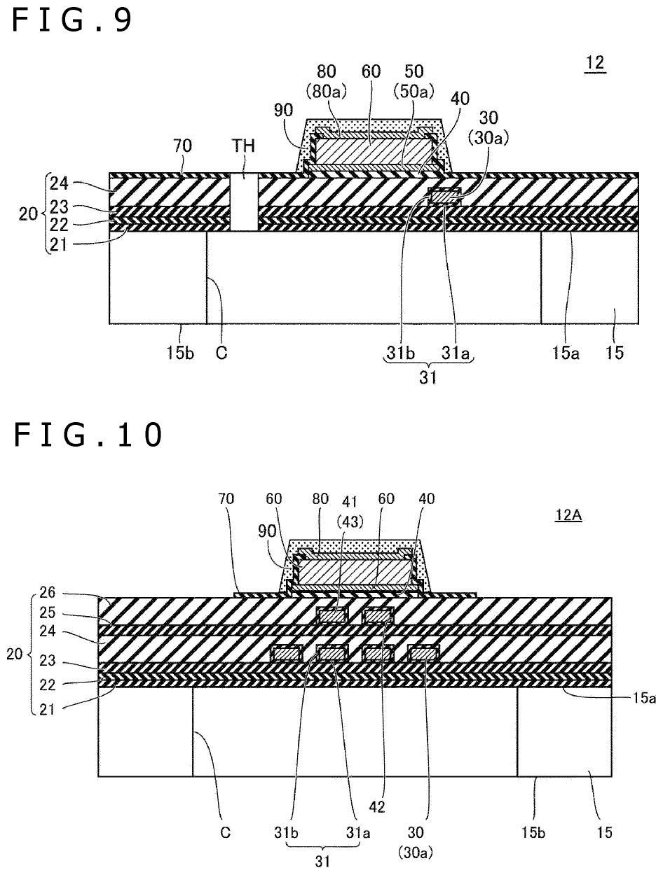

A structure of the humidity sensor 12 will next be described. The humidity sensor 12 has a plurality of layers. As illustrated in FIGS. 7 to 9, the humidity sensor 12 includes a substrate 15, an insulating layer 20, wiring 30, a porous oxide layer 40, a porous metallic layer 50, a solid electrolyte layer 60, an insulating layer 70, a porous metallic layer 80, and a porous protective layer 90. FIG. 7 is a plan view of the humidity sensor 12 in which the porous protective layer 90 is not illustrated. FIG. 8 is a plan view of the humidity sensor 12 in which the insulating layer 70 and the porous metallic layer 80 are not illustrated. FIG. 9 is a sectional view taken along a line IX-IX of FIG. 7. In FIG. 7, the wiring 30 is indicated by a dotted line.

The substrate 15 has a first principal surface 15a and a second principal surface 15b. The first principal surface 15a and the second principal surface 15b are end surfaces in a thickness direction of the substrate 15. The second principal surface 15b is opposite to the first principal surface 15a. A cavity C is formed in the substrate 15. The cavity C penetrates the substrate 15 along the thickness direction of the substrate 15. The cavity C has a rectangular shape as viewed in plan (as viewed from the first principal surface 15a side along the thickness direction of the substrate 15). The substrate 15 contains single crystal silicon, for example.

The insulating layer 20 is disposed on the substrate 15. More specifically, the insulating layer 20 is disposed on the first principal surface 15a. The insulating layer 20 includes, for example, a first layer 21, a second layer 22, a third layer 23, and a fourth layer 24.

The first layer 21 is disposed on the substrate 15 (first principal surface 15a). The first layer 21 contains silicon oxide, for example. The second layer 22 is disposed on the first layer 21. The second layer 22 contains silicon nitride, for example. The third layer 23 is disposed on the second layer 22. The third layer 23 contains silicon oxide, for example. The fourth layer 24 is disposed on the third layer 23. The fourth layer 24 contains silicon oxide, for example.

A portion of the insulating layer 20 which is on the periphery of the cavity C and is disposed on the substrate 15 will be referred to as a peripheral portion 20a. A portion of the insulating layer 20 which is over the cavity C will be referred to as a membrane portion 20b. The membrane portion 20b is formed integrally with the peripheral portion 20a. The membrane portion 20b is thus supported over the cavity C.

The wiring 30 is disposed in the insulating layer 20. More specifically, the wiring 30 is disposed on the third layer 23 and is covered by the fourth layer 24. The periphery of the wiring 30 is covered by a barrier layer 31. The barrier layer 31 ensures adhesion between the insulating layer 20 and the wiring 30. The wiring 30 contains platinum, for example. The barrier layer 31 contains titanium oxide, for example. Note that a portion of the barrier layer 31 which is disposed on the third layer 23 will be referred to as a first portion 31a, and a portion of the barrier layer 31 which covers the wiring 30 will be referred to as a second portion 31b.

The wiring 30 includes a heater portion 30a, an end portion 30b, and a connecting portion 30c. The heater portion 30a is a meandering portion of the wiring 30. The heater portion 30a is disposed in the membrane portion 20b. The end portion 30b is disposed in the peripheral portion 20a. The connecting portion 30c is formed integrally with the heater portion 30a and the end portion 30b to connect them to each other.

The porous oxide layer 40 is disposed on the insulating layer 20. The porous oxide layer 40 contains tantalum oxide, for example. Because the porous oxide layer 40 is porous, the porous oxide layer 40 constitutes a flow passage of gas to be detected by the humidity sensor 12 (detection target gas).

The porous metallic layer 50 is disposed on the porous oxide layer 40. The porous metallic layer 50 contains platinum, for example. The porous metallic layer 50 includes an electrode portion 50a, an end portion 50b, and a connecting portion 50c. The electrode portion 50a is disposed over the membrane portion 20b with the porous oxide layer 40 interposed therebetween, the porous oxide layer 40 having the same shape as the electrode portion 50a as viewed in plan. The end portion 50b is disposed over the peripheral portion 20a with the porous oxide layer 40 interposed therebetween. The connecting portion 50c is formed integrally with the electrode portion 50a and the end portion 50b to connect them to each other. The porous metallic layer 50 is a cathode.

The solid electrolyte layer 60 is disposed on the porous metallic layer 50. More specifically, the solid electrolyte layer 60 is disposed on the electrode portion 50a. The solid electrolyte layer 60 has ionic conductivity. The solid electrolyte layer 60 contains yttria-stabilized zirconia (also referred to as YSZ), for example.

The insulating layer 70 is disposed on the insulating layer 20 to cover the porous oxide layer 40, the porous metallic layer 50, and the solid electrolyte layer 60. However, the insulating layer 70 has an opening formed therein to expose at least part of an upper surface of the solid electrolyte layer 60. The insulating layer 70 includes, for example, a layer containing silicon oxide and a layer containing tantalum oxide which are stacked on top of each other.

A pad portion 30d and a pad portion 50d are arranged on the insulating layer 70. The pad portion 30d is disposed in an opening (not illustrated) which is formed in the insulating layer 20 (fourth layer 24) and the insulating layer 70 to expose the end portion 30b, and is electrically connected to the end portion 30b. The pad portion 50d is disposed in an opening (not illustrated) which is formed in the insulating layer 70 to expose the end portion 50b, and is electrically connected to the end portion 50b.

The porous metallic layer 80 includes an electrode portion 80a, a pad portion 80b, and a connecting portion 80c. The electrode portion 80a is disposed on the solid electrolyte layer 60. The pad portion 80b is disposed over the insulating layer 20 with the insulating layer 70 interposed therebetween. The connecting portion 80c is formed integrally with the electrode portion 80a and the pad portion 80b to connect them to each other. The porous metallic layer 80 is an anode.

The porous protective layer 90 is disposed to cover the porous metallic layer 80 and part of the insulating layer 70. It is sufficient if the porous protective layer 90 covers at least the porous metallic layer 80. The porous protective layer 90 may not cover the insulating layer 70. The porous protective layer 90 has porous portions smaller than siloxane. The porous protective layer 90 can thus capture siloxane on a surface thereof. Accordingly, the porous protective layer 90 prevents siloxane from adhering to the porous metallic layer 80. The porous protective layer 90 contains silicon dioxide or alumina, for example. The porous protective layer 90 is bonded by an oblique deposition method or screen printing, for example.

A through hole TH is formed in the membrane portion 20b and the insulating layer 70 on the membrane portion 20b. The through hole TH is formed in, for example, a U-shape as viewed in plan from a direction normal to the substrate 15, and penetrates the membrane portion 20b and the insulating layer 70 along the thickness direction of the substrate 15. The membrane portion 20b includes a movable portion 20c. The through hole TH is formed on the periphery of the movable portion 20c. The movable portion 20c can thus be displaced along the thickness direction of the substrate 15 with a proximal end of the movable portion 20c as a pivot. The heater portion 30a, the electrode portion 50a, the solid electrolyte layer 60, and the electrode portion 80a are disposed over the movable portion 20c.

The width of the connecting portion 30c of the wiring 30 at the proximal end of the movable portion 20c is larger than the width of the heater portion 30a of the wiring 30.

Now, operation of the humidity sensor 12 will be described. In the following description of the operation of the humidity sensor 12, a case where an oxygen gas in the detection target gas is to be detected will be described by way of example.

When a current is fed through the wiring 30, the heater portion 30a resistively generates heat. Consequently, the solid electrolyte layer 60 is heated and exhibits ionic conductivity. Note that, in a case where the solid electrolyte layer 60 contains yttria-stabilized zirconia, the heater portion 30a heats the solid electrolyte layer 60 to approximately 500° C.

The pad portion 50d and the pad portion 80b are respectively connected to a negative electrode and a positive electrode of a power supply. The detection target gas passes through the porous oxide layer 40 and the electrode portion 50a and reaches an interface between the electrode portion 50a and the solid electrolyte layer 60. The oxygen gas in the detection target gas that has reached the interface between the electrode portion 50a and the solid electrolyte layer 60 becomes oxygen ions by receiving electrons from the electrode portion 50a.

The oxygen ions pass through the solid electrolyte layer 60 and reach an interface between the solid electrolyte layer 60 and the electrode portion 80a. The oxygen ions that have reached the interface between the solid electrolyte layer 60 and the electrode portion 80a release electrons to the electrode portion 80a and become an oxygen gas. Consequently, a current flows between the pad portion 50d and the pad portion 80b. This current is proportional to the concentration of the oxygen gas in the detection target gas. It is therefore possible to measure the concentration of the oxygen gas in the detection target gas by detecting the current.

The concentration of an oxygen gas included in water in the atmosphere can be measured by a similar method. For example, the oxygen gas is electrolyzed at 0.8 V, and water is electrolyzed at 1.2 V. The humidity sensor 12 can electrolyze both oxygen and water by applying 1.5 V to them, for example. Accordingly, a water vapor amount can be determined by obtaining a difference between an oxygen concentration at a time when both oxygen and water are electrolyzed and an oxygen concentration at a time when only the oxygen gas is electrolyzed. The absolute humidity is calculated by dividing the water vapor amount by the mass of dry air.

The controller 100 calculates the absolute humidity from the detection value detected by the humidity sensor 12, and calculates the dew point from the conversion table indicating a relation between the absolute humidity and the dew point temperature, the conversion table being stored in the memory 102 in advance. The controller 100 can thus perform S5 in the processing illustrated in FIG. 6.

The humidity sensor 12 is a YSZ type humidity sensor that includes the solid electrolyte layer 60 containing yttria-stabilized zirconia. The YSZ type humidity sensor is a sensor capable of detecting the absolute humidity and hence obviates a need for temperature correction as compared with a sensor that detects relative humidity. The surface of the humidity sensor 12 is covered by the porous protective layer 90. The porous protective layer 90 has porous portions smaller than siloxane. The porous protective layer 90 can thus capture siloxane on the surface thereof. Hence, the humidity sensor 12 can prevent siloxane from adhering to an inner layer by the porous protective layer 90 and has a good tolerance to siloxane.

### <Modification of Humidity Sensor>

FIG. 10 is a sectional view of a humidity sensor 12A according to a modification. The humidity sensor 12A will be described below focusing on parts different from those of the humidity sensor 12 according to the foregoing embodiment, and description of configurations similar to those of the humidity sensor 12 will be omitted. The humidity sensor 12A is different from the humidity sensor 12 in terms of configurations of the insulating layer 20 and wiring 41. Note that, in the configuration described with reference to FIGS. 7 to 9, the humidity sensor 12 has the through hole TH formed therein. However, the humidity sensor 12A may have no through hole TH as illustrated in FIG. 10 or may have a plurality of through holes TH formed therein. In addition, as illustrated in FIG. 10, the insulating layer 70 may not be disposed on the entire surface of the insulating layer 20.

The humidity sensor 12A is different from the humidity sensor 12 in that the insulating layer 20 additionally includes a fifth layer 25 and a sixth layer 26. The fifth layer 25 contains silicon nitride, for example. The fifth layer 25 is disposed on the fourth layer 24. The sixth layer 26 contains silicon oxide, for example. The sixth layer 26 is disposed on the fifth layer 25.

FIG. 10 is a sectional view taken along a line different from that of FIG. 9 and illustrates a plurality of pieces of wiring 30 in the humidity sensor 12A. However, the wiring 30 of the humidity sensor 12A has the same structure as the wiring 30 of the humidity sensor 12. The humidity sensor 12A also has the wiring 41 disposed in the insulating layer 20. More specifically, the wiring 41 is disposed on the fifth layer 25 and covered by the sixth layer 26. The wiring 41 contains platinum, for example. The periphery of the wiring 41 is covered by a close contact layer 42. The close contact layer 42 contains titanium oxide, for example. The close contact layer 42 ensures adhesion between the insulating layer 20 and the wiring 41.

The wiring 41 includes a temperature sensor portion 43. The temperature sensor portion 43 is disposed in the membrane portion 20b and is a meandering portion of the wiring 41 as viewed in plan. The temperature sensor portion 43 overlaps the heater portion 30a as viewed in plan. The temperature sensor portion 43 functions as a temperature measuring resistor. That is, the temperature in the vicinity of the temperature sensor portion 43 is measured by measuring a change in electric resistance value of the wiring 41 including the temperature sensor portion 43. In this manner, the temperature sensor portion 43 corresponding to the temperature sensor 11 may be formed integrally with the humidity sensor 12A.

### (Summary)

(1)

The hydrogen detection system 1 according to the embodiment of the present disclosure includes the thermal conductivity type hydrogen sensor 10 configured to detect a hydrogen concentration, the humidity sensor 12 configured to detect humidity, and the controller 100. The controller 100 corrects a first detection value detected by the thermal conductivity type hydrogen sensor 10, on the basis of the humidity detected by the humidity sensor 12, and outputs the hydrogen concentration as a second detection value.

(2)

The hydrogen detection system 1 according to (1) further includes the memory 102 as a storage device configured to store information. The memory 102 includes a conversion table that converts the first detection value into the second detection value according to the humidity. The controller 100 converts the first detection value into the second detection value by using the conversion table and outputs the hydrogen concentration.

(3)

The hydrogen detection system 1 according to (1) or (2) further includes the temperature sensor 11 configured to detect a temperature. The memory 102 stores a plurality of tables corresponding to temperatures as the conversion tables. The controller 100 converts the first detection value into the second detection value by using a table corresponding to the temperature detected by the temperature sensor 11 and outputs the hydrogen concentration.

(4)

In the hydrogen detection system 1 according to any one of (1) to (3), the thermal conductivity type hydrogen sensor 10 is able to detect hydrogen concentrations in a range of at least 0.1% to 4%.

(5)

In the hydrogen detection system 1 according to any one of (1) to (4), the humidity sensor 12 is a YSZ type humidity sensor.

(6)

In the hydrogen detection system 1 according to any one of (1) to (5), a surface of the humidity sensor 12 is covered by the porous protective layer 90.

The hydrogen detection system 1 according to the embodiment of the present disclosure includes the thermal conductivity type hydrogen sensor 10 configured to detect a hydrogen concentration, the humidity sensor 12 configured to detect humidity, and the controller 100. The controller 100 corrects the first detection value detected by the thermal conductivity type hydrogen sensor 10, on the basis of the humidity detected by the humidity sensor 12, and outputs the hydrogen concentration as the second detection value. Thus, the hydrogen detection system 1 according to the embodiment of the present disclosure has a high tolerance to siloxane and can suitably detect a wide range of hydrogen concentrations by taking into consideration a detection error caused by the humidity.

The embodiment of the present disclosure has been described above. However, the foregoing embodiment can also be modified in a various manner. In addition, the scope of the present disclosure is not limited to the foregoing embodiment. The scope of the present disclosure is defined by claims and is intended to include all of changes within meanings and a scope equivalent to those of the claims.

With the hydrogen detection system according to the embodiment of the present disclosure, the controller corrects the first detection value detected by the thermal conductivity type hydrogen sensor having a good tolerance to siloxane, on the basis of the humidity detected by the humidity sensor, and outputs the hydrogen concentration as the second detection value. Thus, the hydrogen detection system according to the embodiment of the present disclosure has a high tolerance to siloxane and can suitably detect a wide range of hydrogen concentrations by taking into consideration a detection error caused by the humidity.

What is claimed is:

1. A hydrogen detection system comprising:
a thermal conductivity type hydrogen sensor configured to detect a hydrogen concentration;
a humidity sensor configured to detect humidity; and
a controller, wherein
the controller corrects a first detection value detected by the thermal conductivity type hydrogen sensor, on a basis of the humidity detected by the humidity sensor, and outputs the hydrogen concentration as a second detection value.

2. The hydrogen detection system according to claim 1, further comprising:
a storage device configured to store information, wherein
the storage device includes a conversion table that converts the first detection value into the second detection value according to the humidity, and
the controller converts the first detection value into the second detection value by using the conversion table and outputs the hydrogen concentration.

3. The hydrogen detection system according to claim 2, further comprising:
a temperature sensor configured to detect a temperature, wherein
the storage device stores a plurality of tables corresponding to temperatures as the conversion tables, and
the controller converts the first detection value into the second detection value by using a table corresponding to the temperature detected by the temperature sensor and outputs the hydrogen concentration.

4. The hydrogen detection system according to claim 1, wherein
the thermal conductivity type hydrogen sensor is able to detect hydrogen concentrations in a range of at least 0.1% to 4%.

5. The hydrogen detection system according to claim 1, wherein
the humidity sensor is an yttria-stabilized zirconia type humidity sensor.

6. The hydrogen detection system according to claim 5, wherein
a surface of the humidity sensor is covered by a porous protective layer.
